(19) United States
(12) Patent Application Publication
(10) Pub. No.: US 2008/0167445 A1
(43) Pub. Date: Jul. 10, 2008

(54) ENHANCED OIL RECOVERY COMPOSITIONS COMPRISING PROTEINS AND SURFACTANTS AND METHODS OF USING THE SAME

(76) Inventors: Carl Walter Podella, Irvine, CA (US); John Wilson Baldridge, Newport Beach, CA (US); Andrew Henry Michalow, Mission Viejo, CA (US)

Correspondence Address: Biotechnology Law Group;c/o Portfolioip, P.O. Box 52050, Minneapolis, MN, 55402, US

(21) Appl. No.: 11/969,764

(22) Filed: Jan. 4, 2008

Related U.S. Application Data
(60) Provisional application No. 60/878,412, filed on Jan. 4, 2007.

(51) Int. Cl. C07K 16/00

(52) U.S. Cl. 530/350

(57) ABSTRACT

Disclosed herein are compositions comprising a surfactant and a protein system, where the protein system comprises proteins and stress proteins obtained by the process of fermenting yeast to obtain a fermentation mixture; subjecting the fermentation mixture to stress conditions to obtain a post-fermentation mixture; and centrifuging the post-fermentation mixture and obtaining the supernatant; where the protein mixture retains its functionality under extreme conditions. Also disclosed herein are methods of using the above compositions as enhanced oil recovery agents, cleaning agents or as agents that improve the function of surfactants.

### RELATED APPLICATIONS

The present application claims priority to the U.S. Provisional Patent Application Ser. No. 60/878,412, filed on Jan. 4, 2007, by Podella et al., and entitled “ENHANCED OIL RECOVERY USING SULFATED ALCOHOLS WITH A PROTEIN-BASED SURFACTANT SYNERGIST COMPRISED OF LOW MOLECULAR WEIGHT PROTEINS,” the entire disclosure of which, including any drawings, is hereby incorporated by reference herein.

### FIELD OF THE INVENTION

The present invention is in the field of enhanced oil recovery and cleaning solutions comprising heat and pH stable proteins and peptides and methods of using the same.

### BACKGROUND OF THE DISCLOSURE

There is a critical need in the marketplace to minimize negative toxicological profiles of chemical compositions and formulations, and to increase the use of chemicals based on renewable resources, while at the same time improving performance of the chemical solutions, and achieving those goals with cost effective compounds. The largest uses of chemicals, especially those that are not isolated from the environment, include cleaning and certain industrial processes. Notwithstanding the ecological benefits, adding to the urgency of developing greener chemistries includes regulatory pressure such as the European REACH initiative, which will tightly regulate all chemicals used in high volume in Europe. Further pressure is coming from new exposure data on toxicity of substances, some of which are common and used in households and industry, and the growing liability costs to companies that deal with toxic substances.

### SUMMARY OF THE INVENTION

Disclosed herein are compositions comprising a surfactant and a protein mixture, where the protein mixture comprises proteins and stress proteins obtained by the process of fermenting yeast to obtain a fermentation mixture; subsequently subjecting the fermentation mixture to stress conditions to obtain a post-fermentation mixture; and centrifuging the post-fermentation mixture and obtaining the supernatant; where the protein mixture retains its functionality even under extreme conditions. Also disclosed herein are methods of using the above compositions as enhanced oil recovery agents, cleaning agents or as agents that improve the function of surfactants.

### DETAILED DESCRIPTION OF THE EMBODIMENTS

A key feature that affects the rate and/or efficiency of a chemical process is the surface energy between two or more chemical surfaces, be they liquid-liquid or solid-liquid. Surface energy between two substances is measured as interfacial tension (IFT), and is a function of the two substances. The lower the IFT, the more easily the two surfaces can come into contact. Contact between the two surfaces is a prerequisite for a chemical reaction across the two surfaces to occur. Once the reactants meet, other factors, such as pH, emulsification qualities, reaction energies, temperature, critical micelle concentration, and the like, come into play to affect the rate of chemical reactions.

Typically, a cleaning solution is designed to lower the IFT between the cleaning solution and the “dirt” layer, normally an oily surface, to allow the cleanser within the cleaning solution to come into contact with various components in the “dirt” layer and affect the cleaning. For this reason, enhanced oil recovery and most cleaning solutions comprise a surfactant that lowers the IFT. Some of the currently used cleaning solutions also comprise enzymes that assist with the cleaning.

In many instances, to maximize cleaning efficiency, especially to be effective in removing oily and greasy soils, a high alkaline or high pH solution is useful. See, for example, U.S. Pat. Nos. 6,025,316, 6,624,132, 7,169,237, and U.S. Patent Application Publication No. 20030078178, all of which are incorporated by reference herein in their entirety. In some industrial applications, such as textile cleaning, the sizing agents are removed by cleaning solutions that can exceed a pH of 10. In paper and pulp processing high pH conditions are needed in several steps in the process. At the other end of the spectrum, it may be necessary to use solutions having lower pH, i.e., under acidic conditions, for use in applications such as removal of mineral scale deposits in bathrooms, industrial equipment, cooling systems and the like.

It is also well-known that the use of hot cleaning solutions, such as the use of hot water with the cleaning agent, is desirable under some conditions. Hot solutions can solubilize oils and minerals better.

However, proteins are generally not stable or functional in extreme heat or pH, whether acidic or basic. Proteins are generally functional at a limited temperature and pH range. Outside of the range the protein first loses activity and then becomes denatured. A denatured protein in a cleaning solution is not active and is not useful as a cleaning agent.

Thus, in the first aspect, disclosed herein is a cleaning composition comprising proteins and surfactants, where the proteins retain their stability and functionality under extreme conditions. Extreme conditions can include conditions such as temperatures above about 50° C. and up to about 110° C., steam, pH levels below about 3.5, and pH levels above about 9.5, such as those found in enhanced oil recovery operations. In some embodiments, the pH levels are above 10.5. The proteins of the disclosed compositions comprise proteins, protein fragments, peptides, and stress proteins having a size less than 30 kDa. In some embodiments, the size range is from about 0.5 kDa to about 30 kDa. Throughout the present disclosure, the protein mixture used in the compositions disclosed herein is referred to as the “protein system”.

The word “peptide” includes long chain peptides, such as proteins and enzymes, as well as short chain peptides, such as dimmers, trimers, oligomers, and protein fragments. In some embodiments, the words “peptide” and “protein” are interchangeable. Thus, the protein systems disclosed herein can contain only short chain peptides, only long chain peptides, or a combination thereof.

In some embodiments, by “retaining stability and functionality” it is meant that after being submitted to the extreme conditions the proteins retain at least about 80% of their functionality as compared with the proteins before being submitted to the extreme conditions. In other embodiments, the proteins retain at least about 90% of their functionality after being submitted to the extreme conditions, while in yet other embodiments, the proteins retain at least about 95% of their functionality. Functionality can be defined in terms of the rate of catalysis of a chemical reaction, uncoupling of biochemical processes, lowering of interfacial tension, or lowering of critical micelle concentration.

In some embodiments, the protein systems disclosed herein are derived from an aerobic fermentation of Saccharomyces cerevisiae, which, when blended with surface active agents or surfactants, enhance multiple chemical functions, at ambient conditions, or during and after exposure to the extreme conditions. The protein systems disclosed herein can also be derived from the fermentation of other yeast species, for example, kluyveromyces marxianus, kluyveromyces lactis, candida utilis, zygosaccharomyces, pichia, or hansanula.

After the aerobic fermentation process a fermentation mixture is obtained, which comprises the fermented yeast cells and the proteins and peptides secreted therefrom. In some embodiments, the fermentation mixture can be subjected to additional stress, such as overheating, starvation, oxidative stress, or mechanical or chemical stress, to obtain a post-fermentation mixture. The additional stress causes additional proteins to be expressed by the yeast cells and released into the fermentation mixture to form the post-fermentation mixture. These additional proteins are not normally present during a simple fermentation process. The additional proteins are known as “stress proteins,” and are sometimes referred to as “heat shock proteins”. Once the post-fermentation mixture is centrifuged, the resulting supernatant comprises both the stress proteins and proteins normally obtained during fermentation. The compositions described herein comprise stress proteins.

Several, rather low molecular weight proteins can be produced by Saccharomyces cerevisiae during fermentation as practiced by those familiar in the art. These proteins appear when the yeast cells have been placed under stress conditions during or near the end of the fermentation process. Although referred to as “heat shock proteins,” the stress conditions can occur during periods of very low food to mass concentrations, or as the result of heat shock or pH shock conditions as described in U.S. Pat. No. 6,033,875, Bussineau, et al., incorporated by reference herein in its entirety. In addition, chemical stress, oxidative stress, ultrasonic vibration and other stress conditions can cause the yeast to express the formation of heat shock proteins, more accurately termed, “stress proteins.”

It has further been found that the protein systems disclosed herein, or Live Yeast Cell Derivative, which is an alcoholic derivative from Saccharomyces cerevisiae produced by the methods set forth on Seperti's U.S. Pat. Nos. 2,239,345, 2,230,478 and 2,230,479, all of which are incorporated by reference herein in their entirety, when coupled with surfactants, produce effects that simulate uncoupled oxidative phosphorylation when added to mixed-culture aerobic processes as demonstrated in U.S. Patent Application Publication No. 2004-0180411. Still further details concerning these processes and materials are described in U.S. Pat. No. 6,699,391. Each of these United States patent documents is hereby incorporated by reference herein in its entirety.

The crude Live Yeast Cell Derivative was further refined utilizing dialysis membranes as set forth in U.S. Pat. No. 5,356,874, Bentley, yielding polypeptides having the molecular weights ranging between 6,000 and 17,000 daltons as determined by SDS-page (Bentley, et. al., (1990)). A key difference is that for the compositions disclosed herein, the isolation of fermentation by-products is not necessary, which makes the process more cost effective.

Use of microbial agents for degradation of substances, includes U.S. Pat. Nos. 4,132,638 (liquid is pre-treated with enzymes, followed by microbial thermophilic degradation of slurry), 4,666,606 (eliminating grease & odors using xeronine, which acts by stimulating metabolism of anaerobic and aerobic bacteria), 4,746,435 (use of aerobic and anaerobic microorganisms to purify water of nitrogen, etc.), 5,484,524 (use of biofilm to digest organic matter and pollutants in an aeration chamber for waste water treatment), 5,599,451 (use of anaerobic and aerobic biotreatment of liquid toxic waste, such as pulp and paper waste water), 6,342,386 (uses a polymer and a micro-organism that is capable of producing proteolytic enzymes as a surface coating for removing biofilm), all of which are incorporated by reference herein in their entirety.

The compositions disclosed herein include a stress protein component used in combination with a surfactant-containing composition—for example, an enhanced oil recovery or a cleaning composition—to improve, increase and enhance the surface-active properties, uncoupling of biological processes during and after exposure to extreme pH and/or elevated temperatures, and improve, increase and enhance the heat stability of the sulfated alcohol and sulfonate surfactants, and other low temperature stability surfactants, contained in the EOR composition. Extreme pH is defined as being below 3.5 and above 9.5. Elevated temperature is defined as up to around 10° C.

The “aerobic yeast fermentation process disclosed herein” is defined as the standard propagation conditions utilized in the production of commercially available baker's yeast as described by Tilak Nagodawithana in “Baker's Yeast Production” and further described below. “Live Yeast Cell Derivative (LYCD) disclosed herein)” is defined as an alcoholic extract obtained from yeast prepared as described below.

The “low molecular weight proteins disclosed herein” are defined as the biologically active polypeptide fraction comprised of a size less than 30,000 daltons, which are obtained from aerobic fermentation processes and LYCD.

The “surfactants disclosed herein” are defined as anionic sulfated alcohols, more particularly branched alcohol propoxylate sulfate surfactants, sulfonate surfactants, and other surfactants as described below.

The “Cleaning Compositions” are defined as surfactant-containing compositions used as detergents, cleaners, degreasers, dispersants, emulsifiers, wetting agents, solubolizers, or any other compositions containing surfactants for the purpose of reducing surface tension, interfacial tension, or solubolizing fats, grease or oil, and other organic compounds, both synthetic and naturally derived.

The present inventor has isolated low molecular weight protein factor from aerobic yeast fermentation processes which, when coupled with surfactants, reduce the critical micelle concentration, surface tension and interfacial tension of surfactants, with further reductions in the critical micelle concentration, surface tension, and interfacial tension observed after exposure to grease and oil. This factor was found to be comprised of four polypeptide fractions ranging in molecular weights between about 6,000 and 17,000 daltons by the results of polyacrylamide gel electrophoresis.

The compositions disclosed herein comprise a yeast aerobic fermentation supernatant, surface-active agents and stabilizing agents. Saccharomyces cerevisiae is grown under aerobic conditions familiar to those skilled in the art, using a sugar source, such as molasses, or soybean, or corn, as the primary nutrient source. Alternative types of yeast that can be utilized in the fermentation process may include: Kluyeromyces maxianus, Kluyeromyces lactus, Candida utilis (Torula yeast), Zygosaccharomyces, Pichia and Hansanula. Those skilled in the art will recognize that other and further yeast strains are potentially useful in the fermentation and production of the low molecular weight proteins, “the protein system.” It should be understood that these yeasts and the yeast classes described above are identified only as preferred materials and that this list is neither exclusive nor limiting of the compositions and methods described herein.

Additional nutrients can include diastatic malt, diammonium phosphate, magnesium sulfate, ammonium sulfate zinc sulfate, and ammonia. The yeast is propagated under continuous aeration and agitation between 30° C. and 35° C. and a pH range of between 5.2 and 5.6 until the yeast attains a minimum level of 4% based on dry weight. At the conclusion of the fermentation process, the yeast fermentation product is centrifuged to remove the yeast cells and the supernatant is then blended with surfactants and stabilizing agents and the pH adjusted to between 4.0 and 4.6 for long-term stability.

In an alternative embodiment, the yeast fermentation process is allowed to proceed until the desired level of yeast has been produced. Prior to centrifugation, the yeast in the fermentation product is subjected to autolysis by increasing the heat to between 40° C. and 60° C. for between 2 hours and 24 hours, followed by cooling to less than 25° C. and centrifugation.

In another embodiment, the fermentation process is allowed to proceed until the desired level of yeast has been produced. Prior to centrifugation, the yeast in the fermentation product is subjected to physical disruption of the yeast cell walls through the use of a French Press, ball mill or high pressure homogenization, or other mechanical or chemical means familiar to those skilled in the art, to aid the release of the intracellular, low molecular weight polypeptides. It is preferable to complete the cell disruption process following a heating, or autolysis stage since the presence of the targeted proteins are induced by a heat-shock response. The fermentation is then centrifuged to remove the yeast cell debris and the supernatant is recovered.

In a third alternative embodiment, the fermentation process is allowed to proceed until the desired level of yeast has been produced. Following the fermentation process, the yeast cells are separated out by centrifugation. The yeast cells are then partially lysed by adding 2.5% to 10% of a surfactant to the separated yeast cell suspension (10%-20% solids). In order to diminish the protease activity in the yeast cells, 1 mM EDTA is added to the mixture. The cell suspension and surfactants are gently agitated at a temperature of about 25° C. to about 35° C. for approximately one hour to cause partial lyses of the yeast cells. Cell lyses leads to an increased release of intracellular proteins and other intracellular materials. After the partial lyses, the partially lysed cell suspension is blended back into the ferment and cellular solids are again removed by centrifugation. The supernatant, containing the protein component, is then recovered.

In another embodiment, fresh live Saccharomyces cerevisiae is added to a jacketed reaction vessel containing methanol-denatured alcohol. The mixture is gently agitated and heated for two hours at 60° C. The hot slurry is filtered and the filtrate is treated with charcoal and stirred for 1 hour at ambient temperature, and filtered. The alcohol is removed under vacuum and the filtrate is further concentrated to yield an aqueous solution containing the stress proteins. This LYCD composition is then blended with water, surfactants and stabilizing agents and the pH adjusted to between 4.0 and 4.6 for long-term stability.

In another embodiment, the heat shock process in the preceding embodiment, includes several stages of agitating and heating, cooling and repeating the cycle, to increase the output of heat shock proteins.

In another embodiment, the LYCD is further refined so as to isolate the active proteins having a molecular weight preferably between 500 and 30,000 daltons, utilizing Anion Exchange Chromatography of the crude LYCD, followed by Molecular Sieve Chromatography. The refined LYCD is then blended with water, surfactants and stabilizing agents and the pH of the composition is then adjusted to between 4.0 and 4.6 to provide long-term stability to the compositions.

The foregoing descriptions provide examples of a protein component suitable for use in the compositions and methods described herein. These examples are not exclusive. For example, those of skill in the art will recognize that the protein component may be obtained by isolating suitable proteins from an alternative protein source, by synthesis of proteins, or by other suitable methods. The foregoing description is not intended to limit the term “protein component” only to those examples included herein.

Additional details concerning the fermentation processes and other aspects of the protein component are described in U.S. patent application Ser. No. 10/799,529, filed Mar. 11, 2004, entitled “Altering Metabolism in Biological Processes,” which is assigned to the assignee of the present application and is hereby incorporated by reference herein in its entirety.

### Surfactants

For enhanced oil recovery and cleaning applications, including hard and soft surface removal of soils, odor control, biofilm control, etc., the range of surfactants is not limiting. Anionic, non-ionic and cationic surfactants can be combined with the protein system optimized based on the interaction of the surfactants and protein system/surfactant composition in the application. These include:

Anionic: Sodium linear alkylbenzene sulfonate (LABS); sodium lauryl sulfate; sodium lauryl ether sulfates; petroleum sulfonates; linosulfonates; naphthalene sulfonates, branched alkylbenzene sulfonates; linear alkylbenzene sulfonates; fatty acid alkylolamide sulfosuccinate; alcohol sulfates.

Cationic: Stearalkonium chloride; benzalkonium chloride; quaternary ammonium compounds; amine compounds; ethosulfate compounds.

Non-ionic: Dodecyl dimethylamine oxide; coco diethanol-amide alcohol ethoxylates; linear primary alcohol polyethoxylate; alkyl phenol ethoxylates; alcohol ethoxylates; EO/PO polyol block polymers; polyethylene glycol esters; fatty acid alkanolamides.

Amphoteric: Cocoamphocarboxyglycinate; cocamidopropyl betaine; betaine derivatives; imidazolines.

In addition to those listed above, suitable nonionic surfactants include alkanolamides, amine oxides, block polymers, ethoxylated primary and secondary alcohols, ethoxylated alkyl phenols, ethoxylated fatty esters, sorbitan derivatives, glycerol esters, propoxylated and ethoxylated fatty acids, alcohols, and alkyl phenols, alkyl glucoside glycol esters, polymeric polysaccharides, sulfates and sulfonates of ethoxylated alkyl phenols, and polymeric surfactants. Suitable anionic surfactants include ethoxylated amines and/or amides, sulfosuccinates and derivatives, sulfates of ethoxylated alcohols, sulfates of alcohols, sulfonates and sulfonic acid derivatives, phosphate esters, and polymeric surfactants. Suitable amphoteric surfactants include betaine derivatives. Suitable cationic surfactants include amine surfactants. Those skilled in the art will recognize that other and further surfactants are potentially useful in the compositions depending on the particular cleaning application.

The EOR compositions described herein include one or more sulfated alcohol and sulfonate surfactants at a wide range of concentration levels. These surfactants are anionic in nature, and may be ethoxylated or propoxylated. Preferred anionic surfactants used in EOR applications include branched alcohols, preferably having an alkyl chain length of from C12 to C17, and having an average propoxy groups in the molecule of between 3 and 8.

Those of skill in the art will recognize that interfacial tension is highly specific with regard to the hydrophobic substrate, and therefore, no one specific surfactant will be superior to all others when working with crude oil of different compositions. For instance, light West Texas crude would best use a shortened chain length surfactant while Indonesian crude would require a longer chain length surfactant for optimal results. However, for the purposes of demonstration, a single refined oil composition was utilized for the illustrations in this application.

While it may be advantageous to combine the branched alcohol propoxylate sulfate surfactants with non-ionic or amphoteric surfactants, benefits utilizing these classes may be limited due to low cloud points. These classes of surfactants include:

Non-ionic: Dodecyl dimethylamine oxide; coco diethanol-amide alcohol ethoxylates; linear primary alcohol polyethoxylate; alkyl phenol ethoxylates; alcohol ethoxylates; EO/PO polyol block polymers; polyethylene glycol esters; fatty acid alkanolamides.

Amphoteric: Cocoamphocarboxyglycinate; cocamidopropyl betaine; betaine derivatives; imidazolines.

It should be understood that these surfactants and the surfactant classes described above are identified only as preferred materials and that this list is neither exclusive nor limiting of the compositions and methods described herein.

In another aspect, disclosed herein is a composition comprising stress proteins, surfactants, stabilizers, and an acid. In some embodiments, the acid is selected from the group consisting of phosphoric acid, citric acid, lactic acid and hydrochloric acid.

In another aspect, disclosed herein is a composition comprising stress proteins, surfactants, stabilizers, and a base. In some embodiments, the base is selected from the group consisting of sodium hydroxide, sodium metasilicate, sodium tripolyphosphate, triethanolamine, monoethanolamine, and morpholine.

In another aspect, disclosed herein is a method of cleaning a surface comprising applying to the surface a composition as described herein under extreme conditions. The extreme conditions can include high temperatures and high or low pH, as described above.

In the context of the present disclosure, “cleaning” is defined by its most fundamental features: the chemical removal, or lifting from a surface, or neutralization, of organic, inorganic and biologically based compounds or entities, that create or lead to: (a) unsanitary conditions, (b) unpleasant aesthetics such as stains and dirt, (c) odors, (d) biofilms, (e) impede or disrupt mechanical, chemical and biochemical processes, or (f) crude oil entrapment in underground mineral deposits.

The protein system/surfactant compositions disclosed herein enhance functionality, i.e., increase the efficiency of the cleaning, at ambient, or normal circumstances, or, during or after exposure to extreme conditions. The enhanced functionality is measured in terms of one or more of: (a) removal of inorganic soils, (b) chemical breakdown and solubilization of fats, oils and greases; (c) creation of additional surface active agents during the breakdown of fats and oils yielding an autocatalytic cleaning synergy; (d) removal of odors caused by urine, feces, vomit, other biological fluids, rotting food, biofilm slime and other sources; (e) removal and control of biofilms; (f) enhanced biodegradability of waste products associated with the cleaning processes; (g) stabilized sulfated alcohols and sulfonate surfactants, and other surfactants, to increase their functionality in EOR applications; or (h) uncoupled biological processes, for example, oxidative phosphorylation to: (i) continue to break down biofilms after active mechanical means have ceased and/or industrial processes have been completed, or (ii) enhance EOR by enhancing microbial metabolism (MEOR) using the increase in output of carbon dioxide, the by-product of uncoupling mechanism. All examples and descriptions of the efficacy of the protein systems disclosed herein relate to the maintenance of performance and stability of the compounds, during or after exposure to the extreme conditions.

Similarly, the function of “cleaning” can be summarized as the removal and/or neutralization of undesirable soils from surfaces. The term “surfaces” can refer to either hard surfaces, such as floors, equipment, shelves, automobiles, minerals in the ground (oil recovery), and the like, or soft surfaces, such as fabrics and textiles, or even cleaning up water itself. Objectionable soils include entities such as, oils and greases, mineral deposits, bacterial and viral substances and their secretions, organic compounds both naturally and synthetically derived, malodorous compounds, and combinations of the above. In summary, dirt or soiling of any type ultimately inhibits aesthetics, visual and olfactory, or mechanical or chemical processes. Soiling further includes substances that act as a breeding ground for microbial growth, be it bacterial, viral, fungal, algae, etc., or their secretions. As examples, cleaning floors and equipment can include biofilm control such as biofilm growth in porous surfaces, in paper processing, in cleaning crossflow membranes such as reverse osmosis, micro-filtration and ultra-filtration, industrial tank cleaning and sanitizing, cooling system cleaning including cooling towers and condensers.

In some embodiments, during, or after exposure to extreme environmental conditions, the protein systems and protein system/surfactant compositions disclosed herein remain stable, maintain functionality, and provide improved performance compared to surfactants alone for cleaning, odor control, biofilm control, industrial chemical processes, and enhanced oil recovery (EOR). In further embodiments, the protein system improves functionality and stabilizes sulfated alcohols and sulfonate surfactants at temperatures that exceed 160° F. In some additional embodiments, the protein system and protein system/surfactant compositions can provide complimentary biological enhancements, specifically the upcoupling of biological processes that greatly improve cleaning, odor control, biofilm control, microbial enhanced oil recovery (MEOR) and the range of functionality is maintained during and after exposure to said extreme conditions.

In some aspects, compositions are provided herein, based on the protein systems that bind to surface-active agents, that during and after exposure to the extreme conditions provide the multiple functionality of the surface-active agents. In other aspects, methods are provided by which the production and yields of the protein systems from Saccharomyces cerevisiae may be enhanced.

The compositions and methods disclosed herein are advantageous for various reasons. Many common surfactants are stable at extreme pH conditions, and at temperatures up to 110° C. and therefore these are not always distinguishing features for choosing between surfactants. Second, the control of biological contaminants, such as biofilms, have traditionally been approached as independent of associated cleaning and chemical processes, though they are in most instances integral and interdependent. Thirdly, the control of odors, especially on hard surfaces, in common household, institutional and industrial products has been generally approached by the addition of biocidal agents to cleaners, or other chemical formulations. Odor control and cleaning using traditional approaches require at least two applications of chemicals, one for each issue. The compositions disclosed herein are chemical formulations that comprise a protein system that marries the biological and non-biological aspects of surfactant chemistry and associated processes, as defined here-in.

The rates of chemical reactions and processes, and therefore, the rate of cleaning as defined above, can generally be greatly improved at elevated temperatures. Further, the chemistries in many processes require extreme pH conditions. Most naturally produced peptidic compounds, such as enzymes, that have been developed for industrial and cleaning processes have limitations in their stability and/or functionality at extreme pH conditions and elevated temperatures. Specialized, high temperature enzymes have been developed, but are even more costly than more common enzymes that are used in laundry detergents.

The protein systems disclosed herein surprisingly show not only stability, but also functionality, in terms of the surfactant synergies and both biological and surfactant effects after exposure to the extreme pH and/or elevated temperatures. Thermal stability was tested with exposure for up to 96 hours at around 100° C.

The ability of the protein system to reduce IFT, and to retain this ability during and after exposure to extreme pH levels and/or elevating the temperature above 50° C., is the common denominator that binds the other features of the composition. The unique characteristics of the protein system/surfactant formulations that are enhanced by the reduction in IFT are as follows: reduction of critical micelle concentration; the conversion of a portion of exposed oils and greases into additional surface active agents in both sterile and non-sterile conditions, which provides an autocatalytic effect; de-volatilizing malodorous compounds; penetration of the protein system into, and continuation of breaking down of, biofilms after mechanical application has ceased by stimulating the resident microflora, which improves cleaning, odor control and reducing overall surfactant usage; reducing IFT of sulfated alcohol and sulfonate surfactants, among others; stabilizing sulfated alcohol, sulfonate surfactants, and other surfactants at temperatures above 160° F., where they typically see a large increase in IFT due to thermal degradation, a critical feature in EOR applications; uncoupling of oxidative phosphorylation as a fundamental mechanism affecting microflora continues after pH conditions and temperatures drop to levels that can sustain growth of the microorganisms.

One example that takes advantage of the many features of the protein system/surfactant compositions, during and after raising the temperature above 50° C., is in floor cleaning in restaurants and food processing plants where food oils are a major floor contaminant. The floors are typically comprised of porous concrete, porous ceramic tiles and/or porous grout lines. The pores fill with oils and other organic contaminants that are nutrients to naturally present microflora, which then create tenacious biofilms within the pores. Surfactants that are used in commercially available floor cleaners do not break down oils. Surfactants generally act to emulsify oils. Therefore, when a cleaned floor is made wet, the oil retained in the pores will rise to the top of the water film, creating a slippery layer on the floor surface. Hot water is recommended in many instances because it provides better cleaning performance. The protein system continues to work during the hot mopping step as well as after the mopping is completed by the uncoupling of microbial metabolism, in the naturally occurring microflora. The organic nutrients continue to be broken down at accelerated rates, eliminating oils in particular, which improves slip resistance and general cleanliness. The pores retain much less contaminant and lead to a safer floor, even when it is wet. Finally, many odors emanate from underneath biofilms, typically due to the presence of anaerobic bacteria. Odor reduction, when using the protein system based composition, is a by-product of the chemical process, especially as in cleaning, utilizing a dual mode method of action. Though the protein system is not a sanitizer, by helping to remove organic materials that would remain as nutrients which could help support microbial growth, the elimination of nutrients leads to a more sanitary environment and could potentially reduce the amount of sanitizers, many of which have toxic elements as their purpose is to kill living cells.

An even further benefit of the broad functionality of the protein systems disclosed herein is the simplification of chemical formulation and chemicals used in a particular process. Oils and other hard to remove soils have been traditionally targeted by adding inorganic builders and caustics to surfactant blends. The protein system helps to reduce the need for builders. At the same time, the protein system provides a formulating roadmap to reducing the toxicological and environmental footprints of chemicals, especially those with which people come into contact every day. Due to the low toxicity of the protein system, the compositions can be used in a wide range of application areas such as healthcare, food processing, pharmaceuticals, making enhanced oil recovery and many industrial uses more environmentally favorable.

The options for surface active agents disclosed herein are not limiting and both botanically derived and synthetic, petroleum derived surfactant systems are enhanced by the protein fraction. Surfactant classes include, anionic, non-ionic, cationic, amphoteric. Though petroleum based surfactants are not ideal from an environmental sustainability standpoint, the addition of the protein system in the surfactant system formulation still has benefits that are not available without the protein system. Those include: (a) reduction in the amount of surfactant needed for a particular process, (b) the breakdown of organics at the point of use and continuing to work by breaking down organics in the discharge stream. Further, the synergism allows for less of the surfactant to be used to achieve the same results as indicated by lower interfacial tension and critical micelle concentrations.

pH

The effects at higher alkalinity, or higher pH, are noted when using the protein systems disclosed herein. The synergies provided by the protein system to the blended surfactant system are evident at the high pH levels. Though raising the pH makes the product more hazardous to the user, and perhaps to the discharge stream, in many instances the necessity to clean overrides the safety issue and proper use protocols must be followed to maintain safety. A high pH cleaner can offset the need for solvents and neutralizing pH can be less of an environmental or wastewater treatment issue than solvents, many of which are volatile organic compounds (VOCs) and many of which have toxic effects.

Again, the synergies provided by the protein systems disclosed herein to the surfactant systems used are maintained at the lower pH levels. In the case of EOR, a wide range of pH values may be encountered, with optimal recovery being obtained from acidic conditions to alkaline pH values to as high as 12-13. At near neutral pH, for optimal safety to the user, the protein systems disclosed herein have shown to be superior to performance to even alkaline and solvent enhanced, alkaline cleaners in many instances.

Since the protein systems disclosed herein are stable after exposure to extreme pH conditions, they keep exerting their effect upon natural microflora, in areas such as drains, sewers and septic systems where pH levels tend to be neutralized somewhat due to dilution. After mechanical application procedures such as wiping and mopping are done, functionality is maintained and the protein systems keep on working as in other conditions described herein. Without being bound by any particular theory, it is presumed that the functionality is mostly due to the uncoupling where the natural microflora work to break down organic compounds including biofilms. Without the protein system, the rate of organic degradation is not sufficient to prevent build-up.

### Enzymes

Efforts to reduce the use hazards and environmental problems caused by traditional cleaners based on caustics and/or solvents such as terpenes and glycol ethers, and other chemical compositions, there has been a lot of research done on enzymes. One target area is the development of neutral pH cleaners. Since surfactants alone, or with minimal use of builders are not very effective at relatively neutral pH's, enzymes have been added to improve performance at neutral pH's, though the performance does not approach that of the traditional caustic or solvent based products. Enzymes which have been used to enhance cleaning efficiencies in particular in the development of neutral pH cleaners, include lipase, protease and amylase. Enzymes have the distinct disadvantage of being relatively unstable, especially with regard to temperature and long-term “shelf-life” stability. This greatly limits the range of applications in which enzymes can be used. See, for example, U.S. Pat. No. 6,624,132, incorporated herein by reference, disclosing that its enzyme formulation retain about 50% of its initial activity at 120° F. for at least about 25 days after forming the composition. The present protein systems are stable under conditions that enzymes generally are not.

An alternative to enzymes is the addition of live bacteria, typically in spore form, stabilized in cleaning formulations until diluted at use. The main purpose of cleaning would appear to be to reduce the biology in an area, so that the addition of bacteria would appear to be contra-indicated. And there is still the limitation of IFT, which is not improved by either enzymes or bacterial spores.

Development of extremozymes, enzymes that are extracted from naturally high temperature environments were developed to counter the temperature stability of lipases, proteases and amylases. The limitation is that they react slowly at low temperature. Further, a key characteristic of enzymes is that they are very narrow in the range of soils they will break down. Enzymes can be expensive, and therefore are used at low levels. Finally, there are issues with allergenecity with enzymes, in general.

In any of the above, enzymes do not react synergistically with surfactants nor do they uncouple metabolic processes as with the protein systems disclosed herein.

### Botanically Derived Compounds

In the interest of developing chemical compounds from renewable resources, to reduce the reliance of petroleum based raw materials, chemical companies have been using a number of botanically derived compounds. Petroleum based chemicals, in general, are hazardous and rely on crude oil, which is not a sustainable raw material.

Botanically derived compounds are typically isolated from their natural state and concentrated. These include compounds such as d-limonene from oranges, extracts from pineapple juice and others, with the intent of replacing petroleum based chemicals.

Botanically derived compounds, however, can be toxic. For example, d-limonene has been shown to be a carcinogen, though it is used in cleaners and personal care products that come in intimate contact with people. Solvents such as d-limonene, though derived from natural sources are VOC's, which negatively affect air quality. Further, elevating the temperature of these cleaning compositions above ambient conditions increases the chance of other hazards such as explosions. In tank cleaning, for example, the elimination of solvents can greatly reduce the chance of explosions.

### Biofilms

Biofilm is a polysaccharide complex, which is created by, and surrounds live microflora of many types, and protects the cells from the surrounding environment. The cells within a biofilm matrix act together to produce the biofilm, along with other products such as occasional toxins, in order to maintain their survival.

Biofilm is a noted problem in many areas. For example, the development of biofilm impairs the ability of biocides to kill bacteria. Second, in water treatment, biofilm formation can clog up filters, membranes, heat exchange surfaces, etc. There are actually dozens of ways that biofilms interact with man-made mechanisms. Many times the formation of biofilm acts to impair that which is important to modern society. Thus, many methods to destroy biofilm have been attempted. The general understanding of biofilm production and breakdown by bacteria and how this is affected by an uncoupling agent requires knowledge of some basic cellular processes, including that of energy production and utilization. Furthermore, in order to understand how the bacterial cells generate and utilize energy requires an understanding of the underlying basic science of cellular respiration.

### Uncoupling of Biological Processes

Disclosed herein are specialized yeast fermentation products, which contain bio-active products. The bio-active products include an ‘uncoupling’ agent(s), the protein system, comprised largely of low molecular weight, stress proteins. The primary goal for these stress proteins is to increase microbial substrate utilization, i.e., nutrient uptake. The nutrient of most concern is the biofilm and the intent is that biofilm is degraded. Substrate utilization, or nutrient uptake, is increased because the uncoupler shuts off the ‘oxidative phosphorylation’ (OP) process, which is the most efficient method by which the microorganisms synthesize ATP, the ultimate form of cellular energy. OP occurs in any or all organisms, which are capable of utilizing oxygen as an electron acceptor, also called aerobic organisms. The process of OP occurs due to the actions of membrane-bound molecules, enzymes, co-enzymes, etc. It involves and requires the transfer of electrons, and protons, down an ‘electron transport chain’, which ends in an oxygen molecule acting as the ultimate electron acceptor. The ultimate, indirect, effect of the electron transport chain is the formation of ATP.

With an uncoupler, secondary, less efficient processes are utilized to synthesize ATP. An uncoupler simply uncouples, or, dissociates the electron transport process from the formation of ATP. In addition, because the uncoupler results in the loss of a proton gradient, there is a continual loss of energy in the form of heat. Therefore, the effect of the uncoupler is two-fold. It results in a dramatic increase in the utilization of substrate, or nutrient uptake. This occurs first because the microorganism is forced to utilize less efficient pathways to produce ATP for general metabolic functions in order to survive. Secondly, there is a continued loss of energy the microorganism needs to continuously add ATP, because of the continual loss of energy, and this replacement ATP is generated by inefficient methods.

A further, key manifestation of the loss of energy is that there is inadequate energy left for the formation of complex proteins that are necessary for the building of polysaccharides, or biofilms, thus preventing the build-up of biofilms. The increase of nutrient uptake is accelerated to the point where existing biofilms become a food source for the microflora, which is the presumed mechanism of removal of biofilms.

### EOR Applications

Experimental results show that the sulfated alcohol and sulfonate surfactants may be preferred candidates for EOR as they can be effective at creating low interfacial tension (IFT) at dilute concentrations, and without requiring an alkaline agent or co-surfactant. In addition, some of the formulations exhibit a low IFT at several percent sodium chloride concentrations, and hence, may be suitable for use in more saline reservoirs. Due to the broad functionality of the protein system on surfactants in many conditions, it would be anticipated that the stabilization of sulfated alcohols would be virtually identical in other classes of surfactants that may have lower thermal stability. Data is provided for sulfated alcohols.

Y. Wu et al (A Study of Branched Alcohol Propoxylate Sulfates for Improved Oil Recovery, Society of Petroleum Engineers, 2005) state that surfactant enhanced oil recovery (EOR) has been investigated for many years, especially starting in the 1970's and 1980's when the technology was put on a scientific basis. Unfortunately, the economic reality of the process performance as experienced in the early field trials largely precluded widespread deployment of this technology. However, the recent surge in crude oil prices has provided new impetus to consider employing chemical EOR.

The basic physics behind the surfactant flooding EOR process is that the residual oil, dispersed as micron-sized ganglia, is trapped by high capillary forces within the porous media. Increasing the fluid flow viscous forces or decreasing the capillary forces holding the oil in place are required before the oil can be pushed through the pore throats and sent on to a production well. The rule of thumb for a successful surfactant flood is that the interfacial tension (IFT) between the crude oil and the aqueous phase needs to be reduced to ultra-low interfacial values, several orders below that of a typical reservoir brine-oil system.

Besides the requirement to achieve a low in-situ IFT, another major factor that determines the technical and economic success of a surfactant flood project is to minimize the depletion of the injected surfactant, with a major sink usually from solid adsorption onto clays in the reservoir.

A wide variety of surfactants have been investigated for their potential efficacy for chemical EOR applications. With this renewed interest in surfactant EOR, there is now the opportunity to investigate surfactants not available or not investigated during this earlier development of chemical EOR technology. Branched alcohol propoxylate sulfates have emerged as an effective type of surfactant for the removal of non-aqueous phase liquids (NAPLs) from near surface, aquifer-contaminated sites. This class of surfactants is limited to near surface applications due to instability at temperatures above 160° F. In some deep-well applications, temperatures can reach temperatures as high as 250° F., thus limiting the scope of use for these highly effective surfactants.

Investigation into the use of yeast fermentation by-products for the purpose of ascertaining the degree to which these compounds affect the efficiency of sulfated alcohol and sulfonate surfactants, more particularly branched alcohol propoxylate sulfates, have resulted in the discovery of a group of low molecular weight, stress proteins, or the “protein systems” disclosed herein that, when combined with surfactants, bind with surfactants, resulting in reduction of the interfacial tension when compared to the interfacial tension achieved when using the surfactants alone. A second feature of combining the protein system with surfactants is an increase in heat stability as measured by the degree of shift in the interfacial tensions of the surfactants. A third feature of combining the protein system with branched alcohol propoxylate sulfate surfactants is to further enhance the efficacy of these surfactants for EOR through lower IFT values. A forth feature of combining the protein system with branched alcohol propoxylate sulfate surfactants is to allow their use in deep well, high temperature applications.

The protein system, comprising low molecular weight (0.5-30 kD), stress proteins, in combination with surfactants, were found to yield a further increase in catabolic rates without the proportional increase in biomass, and an increase in the amount of carbon dioxide respired, thus further defining the active proteins responsible for the uncoupling effect observed in the biological processes evaluated. Studies demonstrate the use of either the protein system or the surfactants alone exhibit little effect on the catabolic or anabolic rates. A synergistic effect is observed when surfactants are combined with the low molecular weight proteins. Further studies demonstrated that the protein system component, when combined with surfactants, altered the nature of a given surfactant by reducing its surface tension, critical micelle concentrations, and especially its ability to convert grease and oil into water soluble materials, thus greatly enhancing the surfactant's cleaning efficacy. Although the protein components disclosed herein are preferably obtained by the foregoing fermentation processes, the components may also be obtained by alternative methods, including direct synthesis or isolation of the proteins from other naturally occurring sources.

The addition of the protein systems disclosed herein to a sulfated alcohol surfactant-containing composition has the effect of improving, increasing, and enhancing the surface-active properties and heat stability of the surfactants contained in the composition. This effect has particular advantages in applications in which heat-stabile, surface-active properties of sulfated alcohol surfactants in compositions are desired, including the enhanced oil recovery and cleaning compositions discussed herein.

### EXAMPLES

### Enhanced Oil Recovery Compositions

The enhanced oil recovery compositions described herein generally comprise a branched alcohol propoxylate sulfate surfactant and a low molecular weight protein component produced from the aerobic yeast fermentation process described in the Detailed Description of the Preferred Embodiments. Surfactants utilized are manufactured and distributed by Sasol North America under the trade name of Alfoterra. Adjunct detergent ingredients may include any of a range of additives that are advantageous for obtaining a desired beneficial property. These may include, but are not limited to sodium carbonate or sodium hydroxide to adjust pH, or EDTA to minimize surfactant adsorption onto the formation substrates such as kaolinite clay.

Effect on Interfacial Tension: The following Alfoterra formulations were evaluated for IFT values determined by the pendant drop method using a Kruss prop Shape Analysis System DSA10 to determine the effects of the low molecular weight component on this class of surfactants. Since IFT is highly specific with regard to the oil composition, the EOR compositions may benefit from tailoring blends of the protein-surfactant compositions to create varied alkyl chain length or levels of propoxylation. As a general rule of thumb, the shorter chain alcohols such as the Alfoterra 123 series will be more effective on the lower molecular weight hydrocarbons, while the longer chain alcohols such as the Alfoterra 167 series will be more effective on the higher molecular weight hydrocarbons. Surfactants evaluated for EOR applications are as follows: Alfoterra 123-4S, Alfoterra 123-8S, Alfoterra 145-4S, Alfoterra 145-8S, Alfoterra 167-4S and Alfoterra 167-7S. All of the Alfoterra products contain 30% active material. The stabilized ferment is based on the following:

The compositions utilizing the above surfactants are as follows:

CONCENTRATION
% By Weight

| COMPONENT | Sample A | Sample B |
|---|---|---|
| Stabilized Protein Component | 0.00% | 12.50% |
| (B Samples Only) | | |
| (Product of fermentation of | | |
| saccharomyces cerevisiae) | | |
| Alfoterra 123-4S | 33.33% | 33.33% |
| or Alfoterra 123-8S | 33.33% | 33.33% |
| or Alfoterra 145-4S | 33.33% | 33.33% |
| or Alfoterra 145-8S | 33.33% | 33.33% |
| or Alfoterra 167-4S | 33.33% | 33.33% |
| or Alfoterra 167-7S | 33.33% | 33.33% |
| Water | 66.67% | 54.17% |
| TOTAL | 100.00% | 100.00% |

The initial interfacial tensions for the above samples were tested against Castrol Motor Oil 10W40 as determined by the pendant drop method using a Kruss prop Shape Analysis System DSA10. The “A” samples denote surfactant alone, while the “B” samples contain the protein component. All samples contained 10% surfactant based on an actives basis, and were diluted with water at a 16:1 ratio. The results are as follows:

| | Average | Test #1 | Test #2 | Test #3 |
|---|---|---|---|---|
| | Interfacial | Interfacial | Interfacial | Interfacial |
| | Tension | Tension | Tension | Tension |
| Sample | mN/m | mN/m | mN/m | mN/m |
| Alfoterra 123-4S “A” | 0.352 | 0.353 | 0.353 | 0.350 |
| Alfoterra 123-4S “B” | 0.303 | 0.303 | 0.302 | 0.303 |
| Alfoterra 123-8S “A” | 0.128 | 0.125 | 0.13 | 0.128 |
| Alfoterra 123-8S “B” | 0.112 | 0.111 | 0.112 | 0.113 |
| Alfoterra 145-4S “A” | 0.22 | 0.221 | 0.222 | 0.218 |
| Alfoterra 145-4S “B” | 0.169 | 0.172 | 0.169 | 0.167 |
| Alfoterra 145-8S “A” | 0.093 | 0.093 | 0.092 | 0.093 |
| Alfoterra 145-8S “B” | 0.067 | 0.066 | 0.066 | 0.069 |
| Alfoterra 167-4S “A” | 0.36 | 0.36 | 0.363 | 0.356 |
| Alfoterra 167-4S “B” | 0.321 | 0.322 | 0.316 | 0.324 |
| Alfoterra 167-7S “A” | 1.701 | 1.703 | 1.703 | 1.697 |
| Alfoterra 167-7S “B” | 0.867 | 0.868 | 0.869 | 0.865 |

As can be seen, interfacial tension values were reduced by 10.8% to as much as 49.1% when using the low molecular weight component with the surfactants versus the surfactants alone. The optimal surfactant for the Castrol Motor Oil standard appeared to be the Alfoterra 145-8S with an IFT of 0.093 mN/m. However, the addition of the low molecular weight protein component further reduced the IFT value to 0.067 mN/m, or 28%.

Since sulfated alcohols in general, and Alfoterra branched alcohol propoxylate sulfate surfactants in particular, are heat stabile only at temperatures at or below approximately 160° F., the Alfoterra samples, both with and without the protein component, were heated to the boiling point (approximately 210° F.) for 3-hours, and the IFT values were again measured by the same method. The results are as follows:

| | Average | Test #1 | Test #2 | Test #3 |
|---|---|---|---|---|
| | Interfacial | Interfacial | Interfacial | Interfacial |
| | Tension | Tension | Tension | Tension |
| Sample | mN/m | mN/m | mN/m | mN/m |
| Alfoterra 123-4S “A” | 0.352 | 0.353 | 0.353 | 0.35 |
| 123-4S “A” Heated | 0.618 | 0.617 | 0.62 | 0.616 |
| Alfoterra 123-4S “B” | 0.303 | 0.303 | 0.302 | 0.303 |
| 123-4S “B” Heated | 0.402 | 0.403 | 0.401 | 0.402 |

The addition of the protein component to the Alfoterra 123-4S, a C12-13 branched alcohol sulfate—4 mole propoxylate, reduced the IFT by 13.92%. The degree by which the IFT will change will shift depending on the type of hydrophobe being presented to the various surfactants. However, when the surfactant and surfactant/protein composition are heated to 210° F. +, the IFT for the surfactant variable increases 75.6% from 352 mN/m to 618 mN/m, while the surfactant protein composition's IFT increased from 303 mN/m to 402 mN/m, or 14.2% higher than the unheated surfactant's value of 352 mN/m.

| | Average | Test #1 | Test #2 | Test #3 |
|---|---|---|---|---|
| | Interfacial | Interfacial | Interfacial | Interfacial |
| | Tension | Tension | Tension | Tension |
| Sample | mN/m | mN/m | mN/m | mN/m |
| Alfoterra 123-8S “A” | 0.128 | 0.125 | 0.13 | 0.128 |
| 123-8S “A” Heated | 0.338 | 0.341 | 0.337 | 0.337 |
| Alfoterra 123-8S “B” | 0.112 | 0.111 | 0.112 | 0.113 |
| 123-8S “B” Heated | 0.168 | 0.17 | 0.166 | 0.167 |

Alfoterra 123-8S is similar to the 123-4S, being an 8 mole propoxylate rather than a 4 mole. The data show a modest 12.5% decrease in IFT for the surfactant/protein composition versus surfactant alone. When both variables were heated, the IFT for the surfactant only variable increased 164.1% from 128 mN/m to 338 mN/m. The surfactant/protein composition, however, increased from 112 mN/m to 168 mN/m, an increase of 50%, but only 31.3% over the 129 mN/m for the unheated surfactant alone

| | Average | Test #1 | Test #2 | Test #3 |
|---|---|---|---|---|
| | Interfacial | Interfacial | Interfacial | Interfacial |
| | Tension | Tension | Tension | Tension |
| Sample | mN/m | mN/m | mN/m | mN/m |
| Alfoterra 145-4S “A” | 0.22 | 0.221 | 0.222 | 0.218 |
| 145-4S “A” Heated | 0.422 | 0.425 | 0.421 | 0.421 |
| Alfoterra 145-4S “B” | 0.169 | 0.172 | 0.169 | 0.167 |
| 145-4S “B” Heated | 0.237 | 0.238 | 0.236 | 0.236 |

Alfoterra 145-4S is a C14-15 branched alcohol sulfate, 4 mole propoxylate that exhibited a 23.2% reduction in IFT when combined with the protein component. When both variables were heated for 3 hours, the surfactant only variable IFT increased 91.8%, from 220 mN/m to 422 mN/m. The surfactant/protein composition increased 40.2% from 169 mN/m to 237 mN/m, however, this heated surfactant/protein composition was only 7.7% higher than the unheated surfactant only variable.

| | Average | Test #1 | Test #2 | Test #3 |
|---|---|---|---|---|
| | Interfacial | Interfacial | Interfacial | Interfacial |
| | Tension | Tension | Tension | Tension |
| Sample | mN/m | mN/m | mN/m | mN/m |
| Alfoterra 145-8S “A” | 0.093 | 0.093 | 0.092 | 0.093 |
| 145-8S “A” Heated | 0.241 | 0.24 | 0.242 | 0.24 |
| Alfoterra 145-8S “B” | 0.067 | 0.066 | 0.066 | 0.069 |
| 145-8S “B” Heated | 0.111 | 0.114 | 0.11 | 0.11 |

The Alfoterra 145-8S, which is a C14-15 branched alcohol sulfate, 8 mole propoxylate, demonstrates the lowest IFT at 0.093 mN/m using the Castrol 10W40 Motor Oil as the substrate. When the protein component is added, however, the IFT decreases 28% to 67 mN/m. When heated, the IFT for both samples increase with the surfactant only variable increasing 159.1%, from 93 mN/m to 241 mN/m. The surfactant/protein composition, however, increased a more modest 65.7%, but in relation to the unheated surfactant sample, increased 0.018 mN/m over the unheated surfactant sample, 0.093 versus 0.111 mN/m.

| | Average | Test #1 | Test #2 | Test #3 |
|---|---|---|---|---|
| | Interfacial | Interfacial | Interfacial | Interfacial |
| | Tension | Tension | Tension | Tension |
| Sample | mN/m | mN/m | mN/m | mN/m |
| Alfoterra 167-4S “A” | 0.808 | 0.805 | 0.806 | 0.813 |
| 167-4S “A” Heated | 1.701 | 1.703 | 1.703 | 1.697 |
| Alfoterra 167-4S “B” | 0.759 | 0.762 | 0.757 | 0.757 |
| 167-4S “B” Heated | 0.867 | 0.868 | 0.869 | 0.865 |

Alfoterra 167-4S, which is a C16-17 branched alcohol sulfate, 4 mole propoxylate, shows a 6.1% reduction for the IFT when the protein component is add, reducing the IFT from 0.808 mN/m to 0.759 mN/m. When heated, the IFT for the surfactant only variable increases 110.5%, from 0.808 mN/m to 1.701 mN/m. The surfactant protein composition, however, increases only 14.2%, from 0.759 mN/m to 0.868 mN/m, and the IFT is only 7.3% higher than the unheated surfactant only variable.

| | Average | Test #1 | Test #2 | Test #3 |
|---|---|---|---|---|
| | Interfacial | Interfacial | Interfacial | Interfacial |
| | Tension | Tension | Tension | Tension |
| Sample | mN/m | mN/m | mN/m | mN/m |
| Alfoterra 167-7S “A” | 0.36 | 0.36 | 0.363 | 0.356 |
| 167-7S “A” Heated | 0.645 | 0.64 | 0.642 | 0.639 |
| Alfoterra 167-7S “B” | 0.321 | 0.322 | 0.316 | 0.324 |
| 167-7S “B” Heated | 0.35 | 0.349 | 0.347 | 0.353 |

Alfoterra 167-7S, which is a C16-17 branched alcohol sulfate, 7 mole propoxylate, shows a 10.8% reduction for the IFT when the protein component is add, reducing the IFT from 0.360 mN/m to 0.321 mN/m. When heated, the IFT for the surfactant only variable increases 79.2%, from 0.360 mN/m to 0.645 mN/m. The surfactant protein composition, however, increases only 9.0%, from 0.321 mN/m to 0.350 mN/m, and the IFT is actually 2.8% lower than the unheated surfactant only variable.

An additional feature is that of the conversion of oil and grease to surface-active materials as described in the 2005 U.S. patent application Ser. No. 11/322,104, wherein the levels of surface-active materials, as measured by the critical micelle concentrations, are significantly increased upon continued contact of the protein/surfactant compositions and the grease or oil substrate. This effect is further accentuated when the contact takes place under non-sterile conditions. As an example, a test of a commercially available Bilge Water Cleaner with a pH value of 12.5, available from West Marine, was tested against a prototype bilge water cleaner using a protein/surfactant composition with a pH value of 4.5. Tests using a Kiruss prop Shape Analysis System DSA10 with diesel fuel as a substrate are as follows:

| | 64:1 West Marine | 64:1 |
|---|---|---|
| | System | Protein/surfactant |
| | in Pond | Composition in |
| | Water | Pond Water |
| | (Diesel) | (Diesel) |
| Pre Grease Exposure Solution Properties | | |
| Surface tension prior to grease drop exposure | 33.05 | 27.40 |
| (mN/m) | | |
| CMC prior to grease drop exposure (ppm) | 94 | 179* |
| Properties and Effects During Grease Exposure | | |
| Initial interfacial tension upon exposure to grease | 1.86 | 0.50 |
| (mN/m) | | |
| Equilibrium interfacial tension with grease (mN/m) | 0.78 | 0.38 |
| Time frame for interfacial tension equilibration | 2000 | 70 |
| (minutes) | | |
| Grease drop volume after 2880 minutes = 48 hours | 1.63 | 0.00 |
| (ul) | | |
| Time frame for grease drop volume equilibration | >2880 | 210 |
| (minutes) | | |
| Post Grease Exposure Solution Properties | | |
| Surface tension of 5.0 ml retain after grease exposure | 29.07 | 27.18 |
| (mN/m) | | |
| CMC after grease drop exposure (ppm) | 55 | 76 |
| Calculated Properties Based on the Data Above | | |
| Concentration of the aqueous retain in terms of | 674 | 1000 |
| converted grease (ppm) | | |
| CMC shift pre versus post grease exposure (ppm) | 41 | 103 |
| Percentage of the 5.0 ul grease converted to solubles | 67.4 | 100.0 |
| (%) | | |
| Percentage of the 5.0 ul grease converted to | 4.1 | 10.3 |
| “surfactant-like” materials (%) | | |
| Percentage of grease that is converted which | 6.1 | 10.3* |
| becomes “surfactant-like” (%) | | |

In this case, the protein/surfactant composition converted 10.3% of the grease to “surfactant-like” materials.

Another feature of the surfactant/protein compositions is that they can increase the production of carbon dioxide in the presence of bacteria. This feature is similar to the effects being sought in the Microbial Enhanced Oil Recovery in which the gas production, and the resulting increase in pressure, will help facilitate the sweeping of the oil in the field toward the oil well. In U.S. Patent Application 20040180411, “Altering the Metabolism in Biological Processes”, the surfactant/protein composition has demonstrated the ability to significantly increase carbon dioxide production. In a controlled experiment using bioreactors and a method for capturing and measuring carbon dioxide production, the surfactant/protein composition increased the carbon dioxide production by 432.9%.

### Test Method

The carbon mass balance studies utilized a sterile Tryptic Soy Broth solution that is inoculated with Polyseed, a proprietary blend of aerobic bacteria normally used for 5 Day BOD tests. Tryptic Soy Broth was chosen as a nutrient because it is completely soluble. Therefore, any suspended solids or particulate matter that develop during the course the study is assumed to be biomass produced as a result of the assimilation of the carbon source. Since it is known that 51% of bacteria is comprised of carbon, one can determine the rate of carbon in the nutrient substrate that is converted to biomass by analyzing unfiltered versus filtered samples for total organic carbon at the beginning of the study, followed by sample analysis at any time(s) during the study.

Carbon mass balance studies were conducted to determine the ability of the compositions disclosed herein to affect shifts in carbon uptake, rate of conversion of carbon to biomass, and respiration of carbon dioxide. The studies were conducted using an Applikon Bioreactor using air that has been sparged through a 1.5N sodium hydroxide solution followed by sparging through 2× deionized water to remove all carbon dioxide from the aeration source. The bioreactor exhaust air is then sparged through a 1.5N sodium hydroxide solution so trap all carbon dioxide created in the bioreactor during the test period.

A Tryptic Soy Broth solution is prepared by adding 72 grams of sterile 10% Tryptic Soy Broth concentrate to 2400 ml of 2× deionized water in a 4 liter beaker. Two capsules of Polyseed inoculum is added to the nutrient solution. The inoculated nutrient is heated and maintained at 30 degrees C., with continuous agitation using a stir bar, and incubated for 14 hours. Prior to transferring the nutrient to the bioreactor, the nutrient solution is filtered through 4 layers of cheesecloth to remove the grain used as a substrate for the dried bacteria in the Polyseed. Two liters if the nutrient solution is charged into the bioreactor. Untreated “Controls” are run as a baseline, and “Treated” samples have 10 ppm of the test composition added to the nutrient.

The bioreactor is then sealed and carbon dioxide-free air is sparged at a feed rate of 0.5 liter per minute while the bioreactor temperature is maintained at 30 degrees C. and the turbine mixer run at 500 RPM for the duration of the test. The exhaust air is sparged through a 1.5M sodium hydroxide solution to capture the carbon dioxide being respired. The nutrient is sampled at 0 hours and again at the conclusion of the study. Filtered and unfiltered nutrient samples are analyzed for total organic carbon.

Bioreactor exhaust air is sparged through 200 ml of 1.5N sodium hydroxide solution. Upon completion of the test, the sodium hydroxide solution is transferred to a beaker and 20 ml of a 3.5N barium chloride solution. The solution is neutralized with 4N hydrochloric acid using a pH meter and a burette to determine the volume of hydrochloric acid solution required to neutralize the solution. This is the value for B. The standardization factor is created by neutralizing 200 ml of 1.5N sodium hydroxide solution with 4N hydrochloric acid using a pH meter and a burette to determine the volume of hydrochloric acid solution required to neutralize the solution. This is the value for S. The amount of carbon respired as carbon dioxide is then calculated using S and B in the following equation: C=6N(B-S) where N=7.5

The Carbon Mass Balance can then be calculated as follows: Carbon Nutrient Consumed=Carbon Biomass Increase+Carbon Respired as Carbon Dioxide.

### Results:

| | | Surf./Prot. | % |
|---|---|---|---|
| | Control | Comp. | Difference |
| Carbon-Based Contaminant Reduction | 175.3 | 328.6 | 87.50% |
| Carbon Respired as Carbon Dioxide | 49.5 | 263.8 | 432.90% |
| Biomass Carbon as Sludge | 142.8 | 76.8 | −46.20% |

### Interfacial Tension Tests at Extreme pH Conditions

We measured surface tensions and interfacial tensions against Castrol Motor Oil 10W40 for 32:1 dilutions of each of five samples using a surfactant system, with and without the stabilized protein component, and adjusted to the following pH values:. pH=1, 2, 7.5 (surfactant system only), 12, and 13. The formulae are as follows:

| | Example 1 | Example 2 |
|---|---|---|
| Stabilized Protein Component | 20.00% | 0.00% |
| Ethoxyleted Linear Alcohol | 7.50% | 7.50% |
| (6.0 E.O.) | | |
| Sodium Lauryl Ether Sulfate | 1.50% | 1.50% |
| (3.0 E.O.) | | |
| Water | 71.00% | 91.00% |
| Total | 100.00% | 100.00% |

ph adjusted to 1, 2, 7.5, 12 or 13 with either phosphoric acid or sodium hydroxide (50% solution).

The surface tensions were determined by the Wilhelmy plate method, and the interfacial tensions were determined by the DuNouy ring method, both using a Kiruss Processor Tensiometer K100. All measurements were made in triplicate. The results of the surface tension studies are shown in Table 1, while the results of the interfacial tension studies are shown in Table 2.

TABLE 1
Surface Tension Data - 32:1 Dilutions

| | Average | Test #1 | Test #2 | Test #3 |
|---|---|---|---|---|
| | Surface | Surface | Surface | Surface |
| | Tension | Tension | Tension | Tension |
| Sample | (mN/m) | (mN/m) | (mN/m) | (mN/m) |
| Example 1 @ pH = 1 | 29.97 | 29.93 | 29.99 | 29.99 |
| Example 1 @ pH = 2 | 29.83 | 29.83 | 29.84 | 29.81 |
| Example 1 @ pH = 12 | 29.88 | 29.87 | 29.87 | 29.89 |
| Example 1 @ pH = 13 | 30.32 | 30.35 | 30.27 | 30.34 |
| Example 2 @ pH = 1 | 30.54 | 30.51 | 30.53 | 30.58 |
| Example 2 @ pH = 7.5 | 28.79 | 28.81 | 28.79 | 28.76 |
| Example 2 @ pH = 13 | 30.96 | 30.96 | 30.97 | 30.94 |

TABLE 2
Interfacial Tension Data - 32:1 Dilutions

| | Average | Test #1 | Test #2 | Test #3 |
|---|---|---|---|---|
| | Surface | Surface | Surface | Surface |
| | Tension | Tension | Tension | Tension |
| Sample | (mN/m) | (mN/m) | (mN/m) | (mN/m) |
| Example 1 @ pH = 1 | 3.42 | 3.40 | 3.42 | 3.44 |
| Example 1 @ pH = 2 | 3.36 | 3.36 | 3.35 | 3.38 |
| Example 1 @ pH = 12 | 3.49 | 3.47 | 3.48 | 3.51 |
| Example 1 @ pH = 13 | 3.92 | 3.93 | 3.91 | 3.92 |
| Example 2 @ pH = 1 | 4.80 | 4.77 | 4.82 | 4.80 |
| Example 2 @ pH = 7.5 | 3.94 | 3.89 | 3.97 | 3.95 |
| Example 2 @ pH = 13 | 5.22 | 5.23 | 5.19 | 5.25 |

### Discussion on pH Data

The pH data are plotted above and seem to indicate that there is a modest rise in both surface and interfacial tension as the extreme pH's are approached, and that the rise is more significant at high pH versus low pH. The data clearly indicate that the protein/surfactant composition is preventing a portion of the surface and interfacial tension increases that might otherwise be seen for the surfactant package of the protein system/surfactant composition alone. A bowl shaped (or even V-shape) curve is observed with minima near the mid-pH range in both data sets, for the protein/surfactant composition as well as for the surfactant only samples just from the data that exist.

All patents, patent applications, and literature references cited in this specification are hereby incorporated by reference in their entirety.

Thus, the compounds, systems and methods disclosed herein provide many benefits over the prior art. While the above description contains many specificities, these should not be construed as limitations on the scope of the invention, but rather as an exemplification of the preferred embodiments thereof. Many other variations are possible.

Accordingly, the scope of the present invention should be determined not by the embodiments illustrated above, but by the appended claims and their legal equivalents.

What is claimed is:

1. A composition comprising a surfactant and a protein system, wherein the protein mixture comprises proteins and stress proteins obtained by the process of
fermenting yeast to obtain a fermentation mixture;
subjecting the fermentation mixture to stress conditions obtain a post-fermentation mixture; and
centrifuging the post-fermentation mixture and obtaining the supernatant;
wherein the protein mixture retains its functionality under extreme conditions.

2. The composition of claim 1, wherein the stress conditions are selected from the group consisting of overheating, starvation, oxidative stress, mechanical stress, and chemical stress.

3. The composition of claim 1, wherein the extreme conditions are selected from the group consisting of high temperature, high pH, and low pH.

4. The composition of claim 1, wherein the high temperature is a temperature greater than 50° C.

5. The composition of claim 1, wherein the high temperature is a temperature lower than 110° C.

6. The composition of claim 1, wherein the high pH is a pH above 9.5.

7. The composition of claim 1, wherein the low pH is a pH below 3.5.

8. The composition of claim 1, wherein the stress proteins range in size from about 5 kDa to about 30 kDa.

9. The composition of claim 1, wherein the functionality of the protein mixture under extreme conditions is at least about 80% of the functionality of the protein mixture prior to submission to extreme conditions.

10. The composition of claim 1, wherein the fermenting step is aerobic fermentation.

11. The composition of claim 1, wherein the surfactant comprises one or more of an anionic surfactant, a non-ionic surfactant, a cationic surfactant, and amphoteric surfactant.

12. The composition of claim 1, wherein the functionality is selected from the group consisting of lowering of interfacial tension, lowering of critical micelle concentration, and uncoupling of biochemical processes.

13. The composition of claim 12, wherein the uncoupling comprises control of biofilms.

14. The composition of claim 13, wherein the control of biofilms comprises prevention, reduction and removal of biofilms.

15. The composition of claim 12, wherein the uncoupling comprises oxidative phosphorylation.

16. The composition of claim 13, wherein the biofilms is on a porous surface.

17. The composition of claim 16, wherein the porous surface is selected from the group consisting of concrete, grout, tiles, porous media, wastewater sludge, and fabrics.

18. The composition of claim 17, wherein the porous media comprise crude oil.

19. The composition of claim 12, wherein the uncoupling comprises the accelerated and increased production of CO₂ as a by-product.

20. The composition of claim 19, wherein the accelerated production of CO₂ is used in enhanced oil recovery to improve microbial enhanced oil recovery (MEOR) process.

21. The composition of claim 1, wherein the composition comprises stress proteins, surfactants, stabilizers, and an acid.

22. The composition of claim 21, wherein the acid is selected from the group consisting of phosphoric acid, citric acid, lactic acid and hydrochloric acid.

23. The composition of claim 1, wherein the composition comprises stress proteins, surfactants, stabilizers and a base.

24. The composition of claim 23, wherein the base is selected from the group consisting of sodium hydroxide, sodium metasilicate, sodium carbonate, sodium tripolyphosphate, triethanolamine, monoethanolamine, and morpholine.

25. A method of improving the functionality of a surfactant system, the method comprising adding stress proteins to the surfactant system, wherein the stress proteins are obtained by the process of
fermenting yeast to obtain a fermentation mixture;
subjecting the fermentation mixture to stress conditions obtain a post-fermentation mixture; and
centrifuging the post-fermentation mixture and obtaining the supernatant;
wherein the stress proteins retain their functionality under extreme conditions.

26. The method of claim 25, wherein the said surfactant system comprises sulfated alcohol surfactants.

27. The method of claim 25, wherein the surfactant system comprises sulfonated surfactants.

28. The method of claim 25, wherein the functionality of the surfactant system is improved by raising the thermal stability of the surfactant system.

29. The method of claim 28, wherein the surfactant system is stable at a temperature in excess of 160° F.

30. The method in claim 25, wherein the functionality of the surfactant system is improved by raising the acidic stability of the surfactant system.

31. The method in claim 30, wherein the surfactant system is stable at pH less than 3.5.

32. The method of claim 25, wherein the functionality of the surfactant system is improved by raising the alkaline stability of the surfactant system.

33. The method of claim 32, wherein the surfactant system is stable at pH greater than 9.5.
